(19) United States
(12) Patent Application Publication
(10) Pub. No.: US 2003/0148268 A1
(43) Pub. Date: Aug. 7, 2003

(54) Novel method to identify targets for antibiotic development

(75) Inventors: Vincent A. Fischetti, West Hempstead, NY (US); Daniel C. Nelson, New York, NY (US); Jutta Loeffler, New York, NY (US)

Correspondence Address: DARBY & DARBY P.C., P. O. BOX 5257, NEW YORK, NY, 10150-5257, US

(73) Assignee: THE ROCKEFELLER UNIVERSITY

(21) Appl. No.: 10/310,656

(22) Filed: Dec. 5, 2002

Related U.S. Application Data
(60) Provisional application No. 60/337,196, filed on Dec. 6, 2001.

(51) Int. Cl. C12Q 1/70; C12Q 1/04

(52) U.S. Cl. 435/5; 435/34

(57) ABSTRACT

The present invention identifies a new approach for antibiotic development. By identifying molecules in the cell wall of bacteria responsible for binding bacteriophage lytic enzymes, the present invention focuses on the pathways for possible antibiotic development. The pathway for the bacterial molecule is critical for bacterial survival and thus serves as a target for antibiotic identification.

### PRIORITY

This application claims priority under 35 U.S.C. § 119 from provisional patent application Serial No. 60/337,196, filed Dec. 6, 2001, which is hereby incorporated by reference in its entirety.

### GOVERNMENT SUPPORT

The research leading to the present invention was supported in part Defense Advance Research Project Agency. Accordingly, the U.S. Government may have certain rights in the invention.

### FIELD OF THE INVENTION

This invention relates to the identification of bacterial cell wall substrates that bind the binding domain of bacteriophage (phage) lysins. Specifically, this invention relates to the identification of novel essential biosynthetic pathways in bacteria, which can be used as targets for antibiotic development.

### BACKGROUND OF THE INVENTION

At the end of a bacteriophage lytic cycle in a sensitive bacterial host, double stranded DNA bacteriophages produce lytic enzymes that digest the cell wall of the host bacterium in order to release the progeny phage. The lytic system consists of a holin and at least one peptidoglycan hydrolase, or “lysin,” capable of degrading the major bonds in the bacterial cell wall peptidoglycan. Despite these common bonds, lysins display unique specificity for the host organism or species. Therefore, in addition to a catalytic domain, it is believed that lysins contain a binding domain specific for additional components of a host cell wall (Garcia et al., Proc. Natl. Acad. Sci. USA. 1988, 85:914-918).

Lysins can be divided into four enzyme classes: endo-β-N-acetylglucosaminidases or N-acetylmuramidases (lysozymes), which act on the sugar moiety; endopeptidases, which act on the peptide cross bridge; or more commonly, an N-acetylmuramyl-L-alanine amidase (amidase), which hydrolyzes the amide bond connecting the sugar and peptide moieties. Typically, the holin is expressed in the late stages of phage infection forming a pore in the cell membrane, allowing the lysin(s) to gain access to the cell wall peptidoglycan resulting in release of progeny phage (for review, see Young, Microbiol. Rev. 1992, 56:430-81). Significantly, exogenously added lysin can lyse the cell wall of bacterial cells, producing a phenomenon known as “lysis from without.”

The virulent C₁ bacteriophage specifically infects group C streptococci producing a very powerful lysin that has been partially purified and characterized (Fischetti et al., J. Exp. Med. 1971, 133:1105-1117). Interestingly, the C₁ phage lysin can cause “lysis from without” with groups A and E streptococci as well as group C streptococci (Krause, J. Exp. Med. 1957, 106:365-384), suggesting that these three streptococcal groups share a common cell wall feature.

Pneumococcal phages are classified in four groups based on their viral families. All contain double-stranded DNA and a cell wall lytic system consisting of a holin that permeabilizes the cell membrane, and either an amidase or a lysozyme, capable of digesting the pneumococcal cell wall (Garcia et al., Microb. Drug Resist. 1997, 3:165-76). Both types of enzymes contain a C-terminal choline-binding domain common to many pneumococcal proteins and an N-terminal catalytic domain. The lytic system allows the virus to escape the host cell after successful replication.

The γ phage of Bacillus anthracis (Inglesby et al., J. Am.. Med. Assoc. 2002, 287:2236-2252; Brown and Cherry, J. Infect. Dis. 1955, 96:34-39) has a highly active, specific lysin termed PlyG. PlyG specifically kills B. anthracis and other members of the B. anthracis ‘cluster’ of bacilli both in vitro and in vivo (Schuch et al., Nature 2002, 418:884-889).

### SUMMARY OF THE INVENTION

Bacteriophage have achieved specific identification of susceptible bacterial targets during their association for millions of years with their bacterial host. Lysins are the tools that bacteriophages use to specifically target pathways essential to bacterial viability. Thus, lysins can be used as an entree into the development for specific, new antibiotics.

These new antibiotics would target essential bacterial pathways responsible for the biosynthesis of the bacterial molecule, e.g., cell wall receptor, for the phage lytic enzymes. Since each bacterium has a specific phage, and the binding domain for each phage lytic enzymes differs for each target bacterium, inhibitors of the biochemical pathway that leads to the production of these bacterial molecules are lead compounds in the search for new antibiotics. This information allows for a more direct approach in the antibiotic discovery process, which, in many cases, is still performed by high throughput analysis of thousands of compounds against more uncertain target bacterial molecules to arrive at a lead molecule.

The present invention provides a method of identifying a bacterial molecule essential for bacterial viability. This method comprises identifying a bacterial molecule that binds a bacteriophage lysin binding domain. The bacteriophage lysin binding domain can be a lytically active lysin. In one embodiment, binding of the bacteriophage lysin to the bacterial molecule results in bacterial lysis or cell wall component lysis.

In one embodiment, the bacteriophage lysin binding domain is from a C₁ bacteriophage and the lysin is C₁ bacteriophage lysin. In a related embodiment, the bacteria is an A, C, or E streptococcus. In another embodiment, the bacterial molecule is a polyrhamnose.

In another embodiment, the bacteriophage is a γ phage of B. anthracis and the lysin is PlyG of γ phage of B. anthracis. In a related embodiment, the bacteria is B. anthracis. In another embodiment, the bacterial molecule bound by lysin is N-acetylglucosamine.

In a specific embodiment, the method includes pretreating bacterial cells with an enzyme to fragment the cell wall and to determine whether the fragments released by the enzyme treatment inhibits binding of the phage lytic enzyme. Inhibition of lysin binding domain binding after enzyme treatment indicates that the enzyme was specific for the bacterial cell wall molecule. In one embodiment, the enzymes are selected from proteases, glycosidases, or other cell wall digestive enzymes. In a specific embodiment, the enzymes are selected from the group consisting of Pronase, protease K, trypsin, L-rhamnosidase, glycosidase or Ac-hexosaminidase.

The method of identifying bacterial molecules also includes competition experiments. In one embodiment, inhibition assays are conducted with competitive inhibitors in solution or suspension. Such competitive inhibitors include bacterial molecules including but not limited to, monosaccharides and cell wall carbohydrate extracts. Alternatively, one can use lectins with known carbohydrate binding specificity to identify bacterial molecules by competing with the lysin for binding lectin. A specific embodiment comprises contacting the bacterial molecule with lysin in buffer containing a monosaccharide, cell wall carbohydrate extract, or lectin. A further embodiment employs extracted group A streptococcus carbohydrates as the cell wall carbohydrate extract. Yet another embodiment employs extracted B. anthracis carbohydrates as the cell wall carbohydrate extract.

The present invention further provides a method for identifying a gene for a product in an essential pathway for bacterial viability. This method involves determining whether mutating a gene results in a defect in a bacterial molecule that binds a bacteriophage lysin binding domain, wherein mutation of such a gene indicates that the gene for a product is an essential pathway. In a preferred embodiment, the gene is involved in synthesis of the bacterial molecule. In one embodiment, the defect in the bacterial molecule is loss of bacteriophage lysin binding activity. In another embodiment, the bacteriophage lysin is bacteriophage C₁ lysin. A preferred embodiment would include a polyrhamnose or a cell wall complex containing polyrhamnose as the bacterial molecule. Further, a preferred embodiment would use an A, C, or E streptococcus polyrhamnose as the polyrhamnose. In yet another embodiment, the bacteriophage lysin would be PlyG and the bacterial molecule is N-acetylglucosamine or a cell wall complex containing N-acetylglucosamine. In yet another preferred embodiment, the bacterial molecule is an N-acetylglucosamine from B. anthracis.

The third aspect of the invention provides a method for identifying a lead molecule effective as an antibiotic. This method entails isolating a gene product (for example, an enzyme) of an essential pathway for bacterial viability, which pathway involves the biosynthesis of a bacterial cell wall molecule responsible for binding the lysin binding domain. An inhibitor of an enzyme in this essential pathway would be a candidate molecule. In a preferred embodiment, the gene product (for example, an enzyme) is involved in synthesis of the essential bacterial molecule. In another embodiment, the bacteriophage lysin is bacteriophage C₁ lysin. A preferred embodiment would include a polyrhamnose or a cell wall complex containing polyrhamnose for the bacterial molecule. Further, a preferred embodiment would use an A, C, or E streptococcus polyrhamnose as the polyrhamnose. In yet another embodiment, the bacteriophage lysin is PlyG lysin. Yet another preferred embodiment would include N-acetylglucosamine or a cell wall complex containing N-acetylglucosamine for the bacterial molecule, and preferably B. anthracis N-acetylglucosamine.

### DESCRIPTION OF THE DRAWINGS

The foregoing and other features of the invention will be more readily appreciated from the following description of an exemplary embodiment taken in conjunction with the accompanying drawings, wherein:

FIG. 1 is a schematic diagram of the method of the invention for identifying essential bacterial molecules, thereby identifying essential pathways, which in turn provide target components for drug discovery.

FIG. 2 is a schematic diagram demonstrating that bacteriophage lysins are composed of an N-terminal catalytic domain from one of four conserved hydrolytic classes. The C₁ lysin belongs to the L-alanine amidase class. The PlyG lysin belongs to the L-alanine amidase class. The C-terminal domain is highly variable among these enzymes and contains the recognition or binding domain which gives the lysin specificity for its host. These domains usually recognize a carbohydrate moiety of the polysaccharide which is attached to the peptidoglycan.

FIG. 3 is a schematic diagram of the nuclear magnetic resonance (NMR)-derived structures of streptococcal surface carbohydrates reported as follows: Group A (Coligan et al., Immunochem 1978, 15:755-60; Huang and Krishna, Carbohydrate Res 1986, 155:193-99), A-Variant (Coligan et al., Immunochem 1978, 15:755-60; Huang and Krishna, Carbohydrate Res 1986, 155:193-99), Group C (Coligan et al., Immunochem 1978, 15:755-60), Group E (Pritchard and Furner, Carbohydrate Res 1985, 144:289-96), and S. mutans serotype f (Linzer et al., Infect. Immun. 1987, 55:3006-10; Pritchard et al., Carbohydrate Res. 1987, 166:123-131).

FIG. 4 is a bar graph showing C₁ bacteriophage lysin activity, as measured by a loss in OD over time, on several streptococcal species. Interestingly, the C₁ lysin not only has a more broad host range than the phage itself, but this lysin shows greater activity on Groups A, A-variant, and E streptococci than Group C, the only known host for the C₁ bacteriophage.

FIG. 5 is a graph demonstrating serovars for sensitivity to lysin. Lysin had activity on some strains of S. mutans but not others (FIG. 4). Results detect activity against the f serovar. The diamond shaped marker indicates 10449 Sero c; the square marker indicates Ingbritt Sero c; the triangle marker indicates OMZ175 Sero f; the x marker indicates V100 Sero e; the asterisk marker indicates B14 Sero e; and the circle marker indicates Kir Sero g.

FIG. 6 is a bar graph showing washed cells (approximately 10⁷/ml) pretreated with the following reagents for 20 min at 37° C. prior to exposure with lysin: Pronase (10 ug/ml), lectin# (10 mg/ml of Solanum tuberosum for A and AV or Dolichos biflorus for C), Ac-hexosaminidase (10 U), or L-rhamnosidase (10 U). Note, the extracted carbohydrate for A and C treated with lectin lost reactivity with respective group specific carbohydrate antisera in a precipitin reaction indicating that the epitope was saturated with lectin. In addition, the precipitin reaction with the AV treated with rhamnosidase was very weak indicating that some group carbohydrate remained. The black marker indicates buffer treatment; the gray marker indicates pronase treatment; the white marker indicates lectin treatment; the speckled marker indicates Ac-hexosaminidase treatment; and the striped marker indicates L-rhamnosidase treatment.

FIG. 7 is a bar graph illustrating lysin pretreated with 20 nM Sugar (GlcNAc for A, L-Rhamnose for AV, or GalNAc for C), or extracted Group A carbohydrate (10 mg/ml). The black marker indicates buffer pretreatment; the gray marker indicates sugar pretreatment; and the white marker indicates Group A carbohydrate pretreatment.

FIG. 8 is an SDS-PAGE gel showing analysis of the purified Pal enzyme. Lane 1, crude extract from DH5a (pMSP11), lane 2, purified Pal after affinity chromatography on DEAE cellulose. Molecular weights, in kDa, are indicated on the left.

FIG. 9 is a bar graph demonstrating in vitro killing of 15 clinical S. pneumoniae strains, 2 pneumococcal mutants and 5 oral streptococcal species in log-phase with 100 U/ml Pal during 30 seconds, expressed as the decrease of bacterial titers in powers of 10. Numbers above “S. pneumoniae” indicate serotypes; bold print designates the 9 most frequently isolated serogroups. Error bars show standard deviation of triplicates. I: intermediate susceptibility to penicillin (MIC 0.1-1.0), R: highly penicillin resistant (MIC³2.0).

FIG. 10 is a bar graph demonstrating that N-acetylglucosamine blocks the lytic effect of PlyG lysin. The X axis indicates concentration (mM) of N-acetylglucosamine and the Y axis indicates colony-forming units (CFU ml⁻¹). Each of the samples had 5 μg of PlyG lysin, expect for the last sample marked “no lysin,” which contained no lysin.

### DETAILED DESCRIPTION

The present invention is based in part, on recognition of a key relationship between bacteriophages and their host bacteria. According to the invention, as a result of a phage's association with bacteria over the millennia and the phage's requirement not to become trapped inside the bacterium, the binding domains of phage lytic enzymes have evolved to target a unique molecule in the bacterial cell wall that is essential for the viability of that organism, making bacterial resistance to these enzymes a rare event. Indeed, for pneumococcal phage lysins, the cell wall receptor for the enzyme is choline, a molecule that is important for pneumococcal viability (Tomasz, Proc Natl Acad Sci USA 1968, 59:86-93). Thus, the pathway for these bacterial cell wall molecules are a target for antibiotic development.

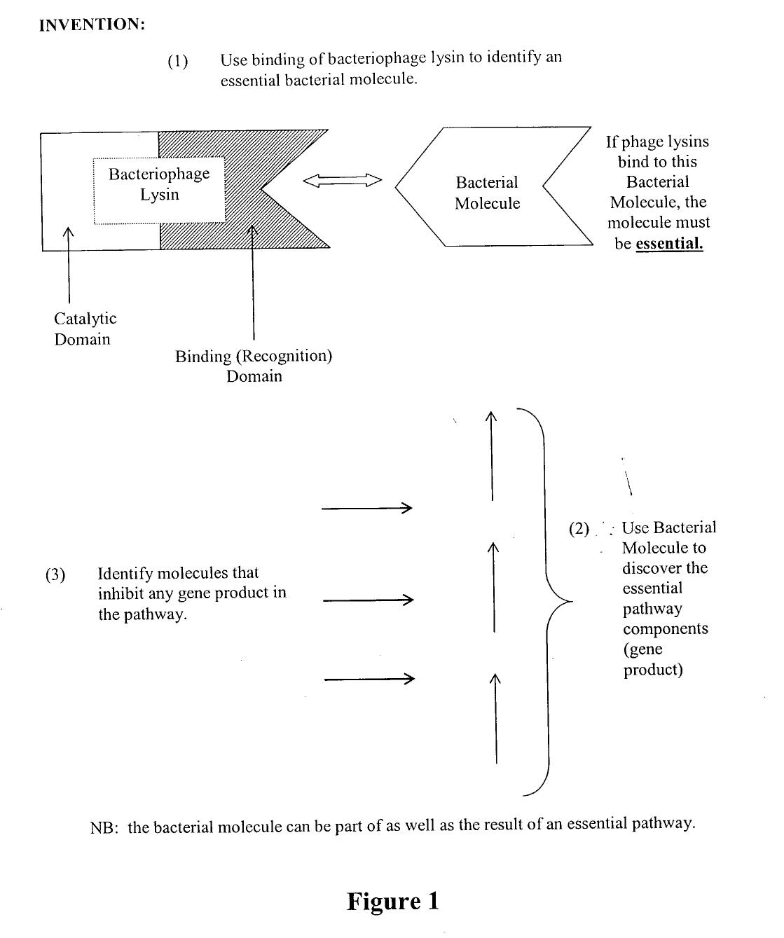

The present invention focuses upon identifying these essential pathways. The method of the invention is shown schematically in FIG. 1. Specifically, the invention identifies the bacterial cell wall molecule of the pathway that is essential to the viability of the bacterium on the basis of its binding to a bacteriophage lysin binding domain (FIG. 1). Known or discovered binding domains of bacteriophage lytic enzyme identify the essential bacterial molecules, which in turn yield their synthetic pathways.

The invention presented here and information generated from other phage lytic enzymes may be used to design new antibiotics. These new antibiotics would target essential bacterial pathways responsible for the biosynthesis of the bacterial molecule, e.g., cell wall receptor, for the phage lytic enzymes. Since each bacterium has a specific phage, and the binding domain for each phage lytic enzymes differs for each target bacterium (particularly Gram-positive bacteria), inhibitors of the biochemical pathway that leads to the production of these bacterial molecules are lead compounds in the search for new antibiotics. This information allows for a more direct approach in the antibiotic discovery process, which, in many cases, is still performed by high throughput analysis of thousands of compounds against more uncertain target bacterial molecules to arrive at a lead molecule. The bacteriophage have achieved specific identification of susceptible bacterial targets during their association for millions of years with their bacterial host.

The approach of the invention advantageously leads to the development of antimicrobials that are pathogen-specific rather than molecules that are broad spectrum.

### Definitions

The terms used in this specification generally have their ordinary meanings in the art, within the context of this invention and in the specific context where each term is used. Certain terms are discussed below, or elsewhere in the specification, to provide additional guidance to the practitioner in understanding the compositions and methods of the invention and how to make and use them.

The term “lysin” as used herein refers to a phage protein that degrades bacterial cell walls. Lysins are one of at least four classes of lytic enzymes encoded by bacteriophages that act on the host cell wall to release progeny phage. These enzymes hydrolyze bonds common to most bacterial cell walls, yet they display unique specificity for the host organism or species.

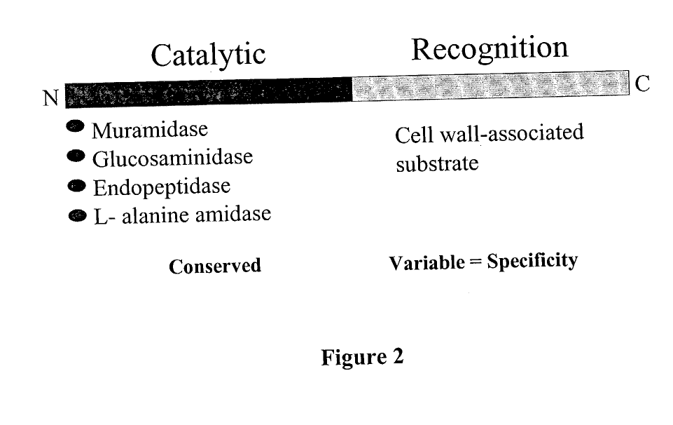

Bacteriophage lytic enzymes are bacterial cell wall hydrolases that are specific for bonds in the bacterial cell wall peptidoglycan. Based on the four major bonds in the peptidoglycan, these enzymes fall into four major classes (muramidase, glucominidase, endopeptidase and N-acetyl-muramyl-L-alanine amidase (amidase)) (FIG. 2). These bacteriophage cell wall hydrolases are generally constructed in two domains, a catalytic domain and a binding domain (FIG. 2). Within an enzyme class, the sequence of the catalytic domains are highly conserved whereas the binding domains are highly variable.

C₁ phage lysin has an apparent molecular weight, as measured by, for example, gel filtration chromatography, of about 100 kDa. Other characteristics of C₁ lysin include strong association with hydroxylapatite (requiring elution with 1 M phosphate); irreversible deactivation by ethylmaleimide, iodoacetamide, and hydroxmercuribenzoic acid; reversible deactivation in dithiopyridine, slight deactivation by iodoacetic acid and no deactivation by sodium tetrathiozine. Lysin is also stabilized (kept enzymatically active) by the presence of reducing agents, including dithiothreitol(DTT), β-mercaptoethanol, cysteine, and glutathione, and in the presence of metal chelators.

PlyG lysin from the γ phage of B. anthracis is another lysin that can be used to identify essential bacterial molecules. The complete nucleotide sequence encoding PlyG is disclosed in GenBank accession #AF536823. This lysin has a molecular mass of about 27,000 and specifically kills B. anthracis isolates and other members of the B. anthracis ‘cluster’ of bacilli in vitro and in vivo (Schuch et al., Nature 2002, 418:884-889).

Purified pneumococcal bacteriophage lytic enzyme (Pal) is yet another lysin that can be used to identify essential bacterial molecules. This lysin has been shown to be able to kill fifteen common serotypes of pneumococci, including penicillin-resistant strains (Loeffler et al., Science 2001, 294:2170-2; Sheehan et al., Mol. Microbiol.1997, 25:717-25; Lopez et al., Microb. Drug Resist. 1997, 3:199-211; Tomasz, Science 1967, 157:694-7).

Table 1 discloses a list of additional lysins which can be used in the present invention; however, the invention is by no means limited to these examples. Table 1 provides the GenBank accession number, name or type of lysin, the bacteriophage name, and the susceptible host organism associated with the lysin.

TABLE 1
Lysins and their Susceptible Host Organisms

| GenBank | | | |
|---|---|---|---|
| Accession | | Bacteriophage | Susceptible |
| Number | Name/Type of Lysin | Name | Host Organism |
| P32762 | N-acetylmuramoyl-L-alanine amidase | HB-3 | Pneumococcus |
| P15057 | Lysozyme (muramidase) | CP-1 | Pneumococcus |
| NP_150182 | N-acetylmuramoyl-L-alanine amidase | MM-1 | Pneumococcus |
| NP_058463 | N-acetylmuramoyl-L-alanine amidase | PVL | Staphylococcus |
| AAB39699 | N-acetylmuramoyl-L-alanine amidase | 80 alpha | Staphylococcus |
| CAA69022 | Cell wall hydrolase, possible endopeptidase | Ply187 | Staphylococcus |
| T13644 | Unknown | phi-Sfi11 | Streptococcus |
| NP_046578 | N-acetylmuramoyl-L-alanine amidase | SPBc2 | Bacillus |
| S25234 | Lysozyme (muramidase) | SF6 | Bacillus |
| CAA72267 | N-acetylmuramoyl-L-alanine amidase | TP21 | Bacillus |
| NP_061527 | Unknown | D3 | Pseudomonas |
| CAA59368 | N-acetylmuramoyl-L-alanine amidase | A511 | Listeria |
| NP_052084 | N-acetylmuramoyl-L-alanine amidase | phiYeO3-12 | Yersinia |
| AAF28126 | Unknown | Unknown | Shigella |
| NP_283954 | N-acetylmuramoyl-L-alanine amidase | Z2491 | Neisseria |
| AAL19962 | Lysozyme (muramidase) | Gifsy-2 | Salmonella |
| NP_458314 | Lysozyme (muramidase) | Unknown | Salmonella typhi |
| BAB19584 | Unknown | Unknown | Escherichia coli |

A bacterium or bacterial cell wall is susceptible to degradation by lysin when, upon contact with a lysin preparation, especially homogeneously purified lysin of the invention, the lysin cleaves the peptidoglycan in the cell wall. Such activity can be measured by measuring optical density (opaque bacteria suspensions become clear, so optical density decreases), immunoassay (for detecting the presence of bacterial molecules released from the inside of the cell wall after lysin treatment, i.e., ATP), or by measuring bacterial viability (lysin activity kills the target or susceptible bacteria), to mention a few such techniques. Other enzyme activity assays are described in the examples.

The term “bacterial molecule that is essential for bacterial viability” as used herein refers to a molecule without which a bacterium is less viable or not viable, has diminished virulence, is less robust, or in some other way is at a survival disadvantage. Such a molecule binds to a bacteriophage lysin binding domain. The bacterial molecule is produced by an essential bacterial pathway. As discussed above, a discovery of the invention is that if a phage lysin binds to the bacterial molecule, it must be essential and therefore disruption of the pathway that produces the molecule is harmful to the bacterium.

The term “essential pathway for bacterial viability” refers to the components, or gene products, of the pathway associated with or that produces a bacterial molecule which is essential for bacterial viability.

The term “bacteriophage lysin binding domain” refers to the site on the bacteriophage lytic enzyme molecule that recognizes the bacterial molecule, as distinct from the lysin catalytic domain. The lysin binding domain can be used independently, as an intact lytically active lysin protein or as part of (in) a fusion protein construct for performing the assays to identify essential bacterial molecules.

The term “polyrhamnose” refers to two or more linked rhamnose molecules. Polyrhamnose acts as a receptor (either alone or compexed with other bacterial cell wall molecules) for C₁ bacteriophage lysin and therefore is also called a “rhamnose receptor.”

The term “gene” in the context of the essential pathways refers to encoding a gene product that is part of the essential pathway. A gene is a sequence of nucleotides which code for a functional gene product. Generally, a gene product is a functional protein, usually an enzyme. However, a gene product can also be another type of molecule in a cell, such as an RNA (e.g., a tRNA or a rRNA). A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

The term “homogeneous” and “homogeneously purified” or any grammatical alternatives (purified to homogeneity, etc.) means that, by suitable analytical testing, including polyacrylamide gel electrophoresis, the preparation is free of impurities, or only contains minor impurities that do not interfere with analytical testing of the preparation, e.g., protein sequencing or enzymatic cleavage, or with the protein's native biochemical activity.

As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). A material shall be deemed isolated if it is present in a cell extract or if it is present in a heterologous cell or cell extract. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

In a preferred embodiment, the method uses purified lysin, lysin binding domain, or a fusion protein containing the lysin binding domain (collectively “lysin”). Any method which results in a homogeneously purified preparation of lysin is embodied within this invention. Lysin produced, for example, by the methods described in the present application, may be removed from other cellular and cultural components. Chromatographic methods of purification are particularly useful embodiments by which to obtain purified protein. Protein purification columns comprising matrices such as hydroxyapatite, anion exchange resins, cation exchange resins, hydrophobic resins and others may be used to purify lysin. A preferred purification matrix for lysin is hydroxylapetite. Size exclusion chromatography and dialysis may also be used to purify these proteins.

Lysin or lysin binding domain can be fused to a tag or a marker. For example, lysin may be fused with an affinity tag such as a polyhistidine tag (e.g. 6 or more histidine residues), a glutathione-S-transferase (GST) tag, a maltose binding tag (malE), a T7 bacteriophage gene 10 peptide tag, a chitin binding domain tag (CBD) or other tags. Alternatively, a lysin fusion with a marker protein such as but not limited to luciferase, green fluorescent protein, alkaline phosphatase, horseradish peroxidase, and the like can be used.

The bacterial proteins discovered in the pathway of the essential bacterial molecule may be expressed by several methods. They may be expressed recombinantly by recombinant bacteria, such as E. coli strains. Protein which is released from a population of infected cells may be collected and used for any other applications embodied by this invention.

General Molecular Biology Techniques and Definitions. Proteins and enzymes are made in the host cell using instructions in DNA and RNA, according to the genetic code. Generally, a DNA sequence having instructions for a particular protein or enzyme is “transcribed” into a corresponding sequence of RNA. The RNA sequence in turn is “translated” into the sequence of amino acids which form the protein or enzyme. An “amino acid sequence” is any chain of two or more amino acids. Each amino acid is represented in DNA or RNA by one or more triplets of nucleotides. Each triplet forms a codon, corresponding to an amino acid. For example, the amino acid lysine (Lys) can be coded by the nucleotide triplet or codon AAA or by the codon AAG. (The genetic code has some redundancy, also called degeneracy, meaning that most amino acids have more than one corresponding codon.) Because the nucleotides in DNA and RNA sequences are read in groups of three for protein production, it is important to begin reading the sequence at the correct amino acid, so that the correct triplets are read. The way that a nucleotide sequence is grouped into codons is called the “reading frame.”

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

A “clone” is a population of cells derived from a single cell.

The term “heterologous” refers to a combination of elements not naturally occurring. For example, heterologous DNA refers to DNA not naturally located in the cell, or in a chromosomal site of the cell. Heterologous DNA may include a gene foreign to the cell. A heterologous expression regulatory element is a such an element operatively associated with a different gene than the one it is operatively associated with in nature. In the context of the present invention, a gene is heterologous to the vector DNA in which it is inserted for cloning or expression, and it is heterologous to a host cell containing such a vector, in which it is expressed, e.g., an E. coli cell.

A “vector” is a replicon, such as plasmid, phage or cosmid, to which another DNA segment may be attached so as to bring about the replication of the attached segment.

A wide variety of host/expression vector combinations (i.e., expression systems) may be employed in expressing the DNA sequences of this invention. Furthermore, expression may occur in either a whole cell or a cell lysate. Cell lysates in which expression may be performed may include rabbit reticulocyte lysate systems. Useful expression vectors, for example, may consist of segments of chromosomal, non-chromosomal and synthetic DNA sequences. Suitable vectors include derivatives of SV40 and known bacterial plasmids, e.g., E. coli plasmids co1 E1, pCR1, pBR322, pMa1-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2 μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. In addition, various tumor cells lines can be used in expression systems of the invention. Host cells which are particularly useful include BL21DE3 E. coli. When transformed with a plasmid bearing a gene which is operably associated with the T7 promoter, the endogenously expressed T7 RNA polymerase causes very high levels of expression of the gene.

### Identification of Essential Bacterial Pathways

The present invention makes it possible to identify essential bacterial pathways using bacteriophage lysins. Thus, the invention advantageously provides a new method for antibiotic development.

The identification of essential bacterial pathways can be achieved in several ways. The bacterial molecule that binds a bacteriophage lysin binding domain can be identified through measuring lytically active intact lysin protein in cell wall or cell wall components (lysin activity indicating binding), or the bacterial molecule that binds a bacteriophage lysin binding domain can be identified through determining inhibition of binding of lysin in the presence of free molecules and targets in solution. Therefore, the initial step is to identify the pathway targeted in the particular bacterial pathogen. In accordance with this invention, one can identify a molecule in such a pathway, and thus the pathway, by determining whether a bacteriophage lysin binds to the molecule in that pathway.

According to the present invention, there are a number of approaches to using a bacteriophage lysin to identify an essential molecule in a bacteria. These include, but are by no means limited to, direct binding assays, particularly with the lysin binding (recognition) domain, and binding inhibition assays, which include enzyme degradation assays (to destroy target bacterial molecules and thereby disrupt lysin binding activity) and competition assays (to inhibit lysin activity with a soluble competitor binding molecule). The detection techniques described for measuring direct binding apply equally to the binding inhibition assays.

Direct binding to cell wall components can be detected by any of the well-known direct binding techniques known in the art, e.g., such as the techniques used in immunoassays for detecting binding of antibody to antigen. In the practice of direct binding, it may be advantageous to use a lysin construct containing the bacteriophage lysin binding (recognition) domain, and omit the catalytic domain. Such a construct can be a fusion construct using a tag, to provide for detection of binding of the recognition domain to the bacterial molecule, e.g., as set forth above. Such a tag may be recognized by an antibody, or it may provide a substrate for direct labeling, e.g., with an enzyme. Suitable enzyme labels include, but are not limited to, luciferase, green fluorescent protein, alkaline phosphatase and horseradish peroxidase. Other labels for use in the invention include colloidal gold, colored latex beads, magnetic beads, fluorescent labels (e.g., fluorescene isothiocyanate (FITC), phycoerythrin (PE), Texas red (TR), rhodamine, free or chelated lanthanide series salts, especially Eu³⁺, to name a few fluorophores), chemiluminescent molecules, radio-isotopes (¹²⁵I, ³²P, ³⁵S, chelated Tc, etc.) or magnetic resonance imaging labels.

Inhibition of binding of a lytic enzyme binding domain to its essential substrate using specific degradation fragments of bacterial cell wall molecules with enzymes provides another approach to identifying the essential bacterial molecules. In this embodiment, bacterial cells or cell walls pretreated with various degrading enzymes are used to determine which fragments block binding. These enzymes may include, but are not limited to, pronase, protease K, trypsin, Ac-hexosaminidase, lipase, other proteases, glycosidases, and glucosidases. Another embodiment would pretreat bacteria with lectin to determine if lysin activity was affected. Another embodiment would pretreat bacteria with Ac-hexosaminidase, which cleaves off terminal GlcNAc and GalNAc residues. In yet another embodiment, bacteria may be treated with L-rhamnosidase to see whether it will dissolve the polyrhamnose backbone of the streptococcal carbohydrate. The possibilities for enzymes can carry over to any relevant enzyme specific for a defined bacterial molecule able to affect bacteriophage activity.

Competition experiments can also be used to determine whether a molecule acts as a lysin binding molecule. In a preferred embodiment, these experiments are conducted with monosaccharides, cell wall carbohydrate extracts in solution, or lectin. In one embodiment, lysin is prepared in buffer containing a final concentration of 20 mM monosaccharide. In another embodiment, extracted group A Streptococcus carbohydrate (10 mg/ml) is used. The ability of a competition molecule, e.g., monosaccharide or cell wall carbohydrate extract, or other bacterial molecule in solution, to inhibit lysin binding indicates that the molecule is part of the bacterial molecule targeted by bacteriophage lysin.

The enzymatic activity of lysin protein cleaves peptidoglycan in the cell walls, e.g., C₁ lysin cleaves peptidoglycan of group A, C and E streptococci. This activity may be measured in vitro, in which aqueous opaque suspensions containing bacteria or isolated cell wall preparations are subjected to lysin enzymatic digestion. The progress of the reaction may be monitored by measuring the optical density of the enzymatic reaction. As the reaction progresses, the optical density decreases. Measurement of the reaction velocity may be performed by calculating the rate of decrease in optical density. Reaction velocity data can indicate whether a particular molecule is a target of the lysin based on direct binding, enzyme degradation, or competition experiments.

Bacterial cell walls may be digested extensively with a variety of cell wall cleaving enzymes (muramidase, glucominidase, endopeptidase or amidase). The resulting fragments may then be separated by chromatographic techniques and the eluted fragments tested for its ability to bind the binding domain of a phage lytic enzyme by one of the assays described above. NMR analysis of the reactive cell wall fragment will allow for the structural composition of the binding fragment.

Once an essential molecule has been identified by virtue of its ability to bind to a bacteriophage lysin binding domain, it is possible to identify potential antibiotic targets in the synthetic pathway of that molecule. Many synthetic pathways for bacterial molecules are known. Others can be determined from bacterial genomes that are becoming available. Still other pathways, and the genes encoding products in these pathways, can be uncovered by analogy to known pathways from other bacteria, e.g., using the powerful techniques of bioinformatics or the well established techniques of molecular cloning based on sequence homology to known genes.

In addition, it may be possible to identify genes and their encoded products by inserting them into host cells and determining whether expression of the gene, or a gene cluster, renders the host cell susceptible to binding by the bacteriophage lysin. Evidence of such binding provides evidence that the inserted gene is part of the pathway of interest. In this embodiment the genome of a phage enzyme sensitive is fragmented into large fragments and inserted using a phagemid into a phage enzyme resistant organism. Organisms that have converted to phage enzyme sensitive have the fragment coding for the binding domain.

### Screening and Chemistry

Any screening technique known in the art can be used to screen for antagonists of the pathways associate with an essential bacterial molecule identified in accordance with the invention. When the pathway has been identified an assay will be performed to measure a product of the reaction in the pathway. The present invention contemplates screens for small molecules and mimics, as well as screens for natural products that bind to the molecules in the screen and block product production.

Knowledge of the primary sequence of the inhibitory polypeptide fragment, and the similarity of that sequence with proteins of known function, can provide an initial clue as to inhibitors or antagonists. Identification and screening of antagonists is further facilitated by determining structural features of the target protein, e.g., using X-ray crystallography, neutron diffraction, nuclear magnetic resonance spectrometry, and other techniques for structure determination. These techniques provide for the rational design or identification of agonists and antagonists.

Another approach uses recombinant bacteriophage to produce large libraries of compounds. Using the “phage method” (Scott and Smith, Science 1990, 249:386-390; Cwirla, et al., Proc. Natl. Acad. Sci. USA 1990, 87:6378-6382; Devlin et al., Science 1990, 49:404-406), very large libraries can be constructed (10⁶-10⁸ chemical entities). A second approach uses primarily chemical methods, of which the Geysen method (Geysen et al., Molecular Immunology 1986, 23:709-715; Geysen et al. J. Immunologic Methods 1987, 102:259-274; and the method of Fodor et al. (Science 1991, 251:767-773) are examples. Furka et al. (14th International Congress of Biochemistry 1988, Volume #5, Abstract FR:013; Furka, Int. J. Peptide Protein Res. 1991, 37:487-493), Houghton (U.S. Pat. No. 4,631,211) and Rutter et al. (U.S. Pat. No. 5,010,175) describe methods to produce a mixture of peptides that can be tested as agonists or antagonists.

In another aspect, synthetic libraries (Needels et al., Proc. Natl. Acad. Sci. USA 1993, 90:10700-4; Ohlmeyer et al., Proc. Natl. Acad. Sci. USA 1993, 90:10922-10926; Lam et al., PCT Publication No. WO 92/00252; Kocis et al., PCT Publication No. WO 9428028) and the like can be used to obtain compounds for screening according to the present invention.

Thus, test compounds are preferably screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle et al., TIBTech 1996, 14:60).

High-Throughput Screen. Inhibitory agents that block essential pathways identified according to the invention may be found by screening in high-throughput assays. It will be appreciated by those skilled in the art that different types of assays can be used to detect different types of agents. Several methods of automated assays have been developed in recent years so as to permit screening of tens of thousands of compounds in a short period of time (see, e.g., U.S. Pat. Nos. 5,585,277, 5,679,582, and 6,020,141). Such high-throughput screening methods are particularly preferred.

### Uses and Benefits

The methods of the present invention will identify key pathways in bacteria, e.g., that are part of cell wall synthesis necessary for bacterial survival or viability, and are thus conserved by the bacteria during evolution. With this knowledge one can employ screening analysis to identify those compounds that inhibit vulnerable pathways, particularly in major pathogens. Such antibiotics offer targeted killing of pathogens without affecting normal flora bacteria or the host organism.

### Antibodies

According to the invention, one possible approach to antibacterial therapy would be to develop active or passive immunotherapy targeted to essential bacterial molecules identified in accordance with this invention. Molecules produced recombinantly or by chemical synthesis, and fragments or other derivatives or analogs thereof, including fusion proteins, may be used as an immunogen to generate antibodies that recognize the essential molecules. Such antibodies include but are not limited to polyclonal, monoclonal, chimeric, single chain, Fab fragments, and an Fab expression library. The antibodies of the invention may be cross reactive, e.g., they may recognize molecules from different bacterial species. Polyclonal antibodies have greater likelihood of cross reactivity.

Various procedures known in the art may be used to generate polyclonal antibodies to essential bacterial molecules, whether for active or passive vaccines. For the production of antibody, various host animals or patients can be immunized by injection with the molecule, or a derivative (e.g., fragment or fusion protein) thereof, including but not limited to human patients, rabbits, mice, rats, sheep, goats, etc. In one embodiment, the molecule can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immnunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

### EXAMPLES

The following Examples illustrate the invention, but are not limiting.

### Example 1

### The C₁ Bacteriophage Lysin Binding Domain Recognizes an Alternating (α1→2) and (α1→3) Polyrhamnose Backbone in Streptococcal Cell Wall Carbohydrates.

This example takes advantage of the limited range of streptococci susceptible to the C₁ bacteriophage lysin. Although bacteriophages have long been known to contain lytic enzymes, the specificity of these enzymes for a particular range of organisms has not been extensively investigated, with two notable exceptions. In pneumococcus, choline has been well characterized as a unique constituent of all pneumococcal cell walls (Tomasz, Science 1967, 157:694-97). Interestingly, the pneumococcal bacteriophage lysins, as well as autolysins, usually contain choline binding domains. Fusion proteins have been made with either the Clostridium acetobutylicum lysozyme or Lactococcus lactis phage lysozyme catalytic domains and the pneumococcal choline binding domain (For review, see Lopez et al., Microb. Drug Resist. 1997, 3:199-211). These chimeras retain both the lytic activity and the dependence on choline. This “modular” design of lysins gave arise to the current proposed structure of these enzymes as depicted in FIG. 2. However, it was not known if these recognition or binding domains for the lysins was unique to the pneumococcal system or if it could be extended to other organisms. This question was partially answered by Loessner et al., who showed that a cloned Listeria monocytogenes phage lysin had activity against 64 serovars of Listeria, yet had no activity against almost 40 other bacterial species tested (Loessner et al., Mol. Microbiol. 1995, 16:1231-41). These results support the theory that lysins have evolved domains which recognize conserved receptors specific to the host range.

Only a limited range of streptococci are susceptible to the C₁ bacteriophage lysin. Here it is shown that the basis for this specificity is recognition by the lysin of an alternating (α1→2) and (α1→3)-linked polyrhamnose backbone.

The rhamnose biosynthesis pathway is found in bacteria and plants, but is not found in humans. Bacteria with deletions in the rhamnose biosynthesis pathway have been shown to be severely defective in their ability to cause disease (Xu, Infect. Immun. 2000, 68:815-23; Yamashita et al., Infect. Immun. 1999, 67:3693-97). Because of this, the rhamnose biosynthetic pathway has been targeted for antibacterial development (Giraud and Naismith, Curr. Opin. Struc. Biol. 2000, 10:687-96). The group A streptococcal results described in this example, in which the C₁ phage lytic enzyme binds to polyrhamnose, independently establish that the rhamnose synthetic pathway should be targeted for antibacterial development.

### Methods

Identification of rhamnose as a C₁ bacteriophage lysin receptor. Three groups of washed cells were divided into Group A Streptococcus, Group A-Variant, and Group C Streptococcus. Each group was pretreated with the following reagents for 20 minutes at 37° C. prior to exposure with lysin: Pronase (10 μg/ml), lectin^{#} (10 mg/ml of Solanum tuberosum for A and AV or Dolichos biflorus for C), Ac-hexosaminidase (10 U), or L-rhamnosidase (910 U).

Competition experiments. The three groups of cells were pretreated with monosaccharide or buffer containing 20 mM monosaccharide (GlcNAc for A, L-rhamnose for AV, or GalNAc for C), or extracted Group A carbohydrate (10 mg/ml) prior to exposure to the C₁ lysin.

### Results

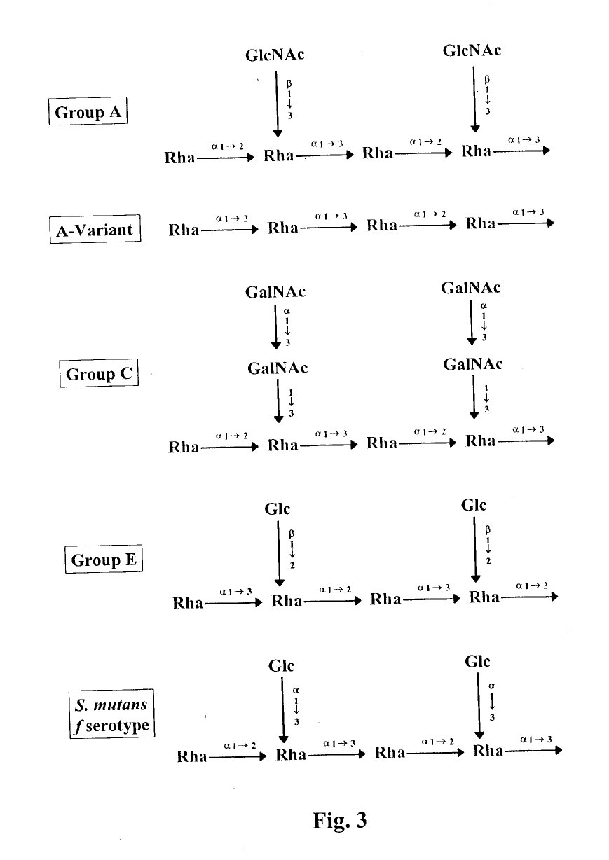

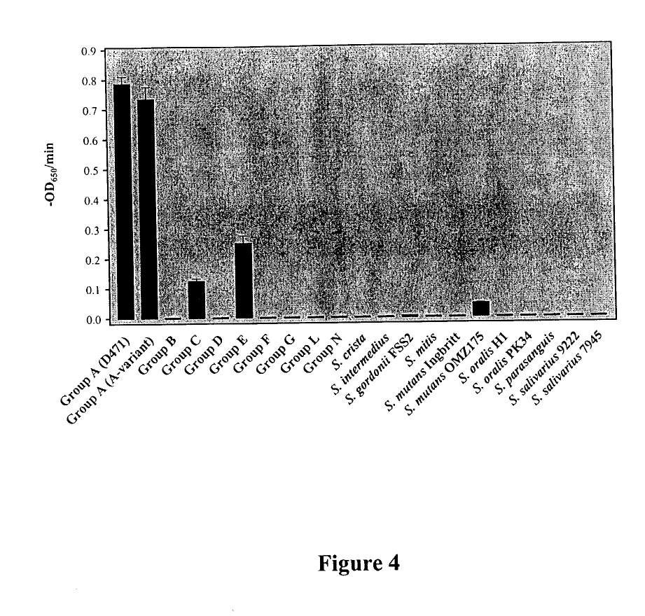

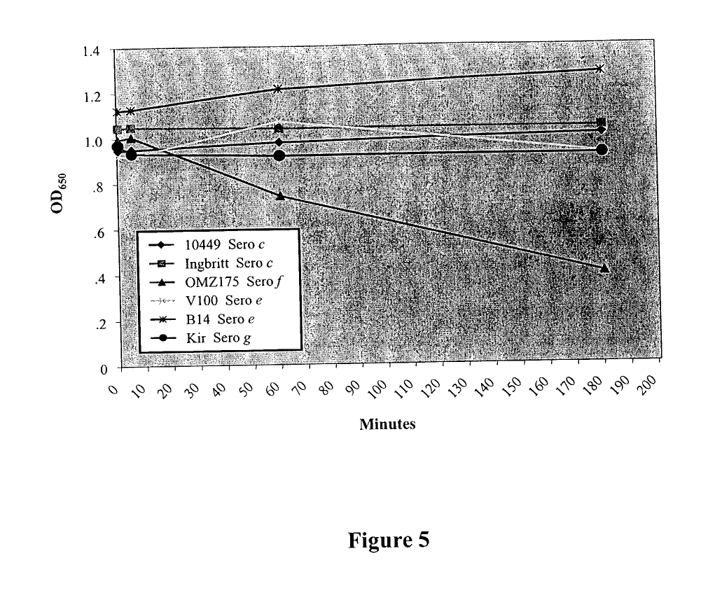

To establish polyrhamnose as a potential receptor for the C₁ bacteriophage lysin, a series of assays using cells that had been pretreated with modifying enzymes or free binding molecules were undertaken. Protease treated cells remained sensitive to lysin, which excluded the possibility of a proteinaceous receptor. NMR studies of the carbohydrate for Groups A, C, and E are known (FIG. 3), and initial inspection of these structures indicated that a backbone of alternating (a 1→2) and (a 1→3) linked rhamnose residues is the only conserved structural component between these three species (Coligan et al., Immunochem. 1978, 15:755-760; Huang and Krishna, Carbohydrate Res. 1986, 155:193-9; Pritchard and Furner, Carbohydrate Res. 1985, 144:289-96). Further support for the polyrhamnose backbone as being a lysin receptor is indicated by the ability of lysin to hydrolyze the Group A-Variant (AV) strain, which lacks the streptococcal group-specific side chains and consists solely of the polyrhamnose backbone (Coligan et al., Immunochem. 1978, 15:755-60; Huang and Krishna, Carbohydrate Res. 1986, 155:193-99). A wider examination of Gram-positive bacteria identified Streptococcus mutans strain OMZ175 as susceptible to the lytic actions of lysin, in addition to the aforementioned strains (FIG. 4). Testing of several S. mutans species revealed that only the f serotype of OMZ175 was hydrolyzed by lysin (FIG. 5). Significantly, this species has the identical polyrhamnose backbone observed in groups A, C, and E streptococci (FIG. 3) (Linzer et al., Infect. Immun. 1987, 55:3006-10; Pritchard et al., Carbohydrate Res. 1987, 166:123-31).

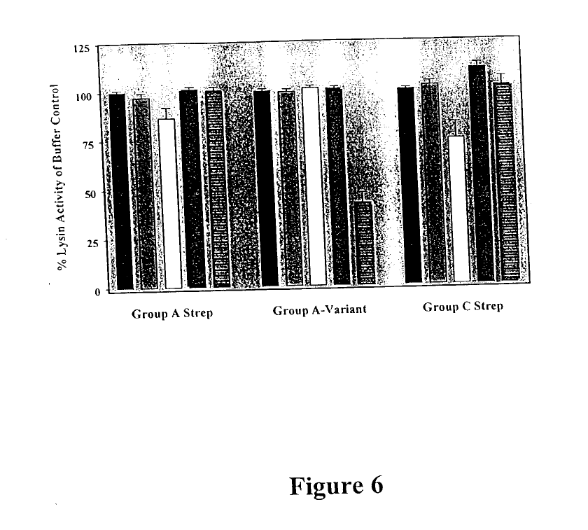

In enzyme pretreatment experiments with Groups A, AV, and C streptococci (FIG. 6), pretreatment of cells (about 10⁷/ml) with pronase (10 μg/ml) had no effect on the lytic ability of lysin for these cells. This finding is consistent with our previous results using protease K and trypsin, and suggests that the C₁ bacteriophage lysin receptor is not of a proteinaceous origin. Thus, additional studies were carried out on these three strains to investigate any contribution of the carbohydrate.

When cells were pretreated under the same conditions with lectin (100 μg/ml of Solanum tuberosum for groups A and AV or Dolichos biflorus for group C), no decrease in lysin activity was observed. The S. tuberosum lectin is specific for the N-acetyl-glucosamine of the group A streptococcal carbohydrate and the D. biflourus is specific for the N-acetyl-galactosamine of the group C streptococcal carbohydrate. It was found that the extracted carbohydrate from groups A and C streptococci that were treated with lectin had lost reactivity with respective group-specific antisera in a precipitin reaction indicating that the lectin was at, or above, saturation levels for its epitope. In a complementary experiment to rule out any side chain contributions to the binding, cells were pretreated with Ac-hexosaminidase (10 U), which would specifically cleave off terminal GlcNAc and GalNAc residues. Once again, lysin activity was not diminished and we concluded that the group-specific side chains, which are the basis for serological reactivity, do not contribute to the binding of lysin.

Finally, we pretreated cells with L-rhamnosidase (10 U) in an attempt to dissolve the polyrhamnose backbone of the streptococcal carbohydrate. In this experiment, lysin had no effect on groups A or C streptococci. We presume this is due to the side chains hindering the terminal (exo) rhamnosidase activity of the enzyme. However, in the AV strain, we did see a greater than 50% decrease in lysin activity. Precipitin reactions with the AV carbohydrate treated with rhamnosidase was very weak, indicating that while we had removed most of the carbohydrate, some remained.

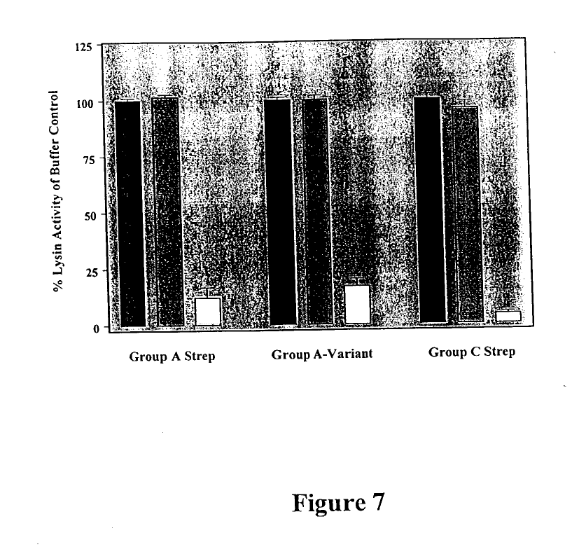

To determine the specificity of rhamnose as a lysin receptor, we performed competition experiments with monosaccharides or cell wall carbohydrate extracts (FIG. 7). When cells were pretreated with monosaccharide or lysin was used in buffer containing a final concentration of 20 mM monosaccharide (GlcNAc for group A cells, L-Rhamnose for AV cells, or GalNAc for group C cells), no decrease in lysin activity was observed. The results for the GlcNAc and GalNAc were expected as we had shown above that the side chain does not contribute to the lysin specificity; however, the fact that the L-rhamnose had no effect indicated that lysin requires more than a single rhamnose residue for binding. In similar competition experiments using extracted group A carbohydrate (10 mg/ml), lysin activity was significantly reduced greater than 75%.

Thus, in summary, the group A carbohydrate contains polyrhamnose with GlcNAc side chains and the AV carbohydrate contains only polyrhamnose without the side chains. Because both are cleaved with the C₁ lysin, the GlcNAc side chain must not play a role in the binding activity, leaving the polyrhamnose as the likely binding substrate. Since L-rhamnose does not block enzyme activity but the intact AV carbohydrate does, we assume that polyrhamnose (two or more linked rhamnose molecules) is the substrate for the C₁ enzyme. Therefore, lysin appears to be specific for polyrhamnose of (Rha)n, where n is at least 2.

### Example 2

### Pneumococcal Bacteriophage Lytic Enzyme

This Example focuses upon a purified pneumococcal bacteriophage lytic enzyme (Pal). In the present example it is demonstrated that seconds after contact, a purified Pal enzyme is able to kill 15 common serotypes of pneumococci, including penicillin-resistant strains. Furthermore, it is demonstrated that Pal-resistant pneumococci could not be detected after extensive exposure to the enzyme. This finding confirms that the enzyme binds a molecule essential to pneumococci.

### Methods and Results

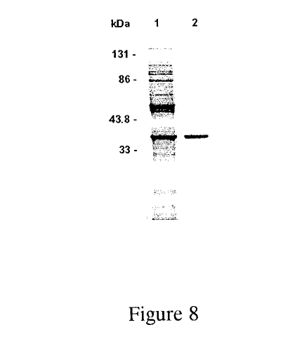

E. coli DH5α (pMSP11) expressing the amidase Pal of phage Dp-1 (Sheehan et al., Mol Microbiol 1997, 25:717-25) was obtained. The enzyme was produced in E. coli and purified by affinity chromatography in a single step as described, with some modifications (Sanchez-Puelles et al., Eur J. Biochem 1992, 203:153-9) (FIG. 8). We defined a unit for the enzyme using lysis of exponentially growing S. pneumoniae serogroup 14 with serial dilutions of purified Pal. The purification process yielded an average of 15 U of enzyme per μg protein.

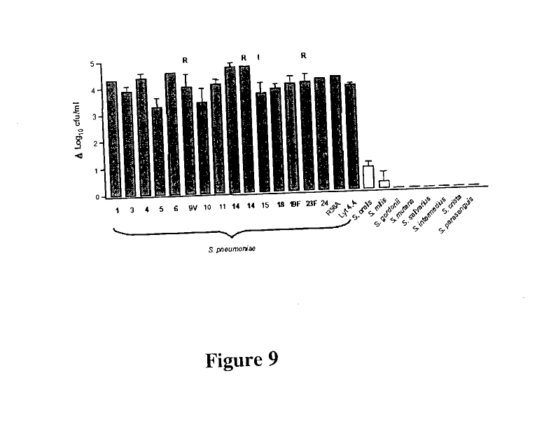

The first series of experiments measured the killing ability of Pal in vitro by exposing 15 clinical strains of S. pneumoniae, 2 pneumococcal mutants (R36A, Lyt 4-4) and 5 species of oral commensal streptococci (S. gordonii, S. mitis, S. mutans, S. oralis, S. salivarius) to purified enzyme at a final concentration of 100 U/ml, and in the case of the oral streptococci to 1,000 and 10,000 U/ml. The pneumococcal strains, obtained from various sources (Table 2), included 9 serogroups that most frequently cause invasive disease in North America, Europe, Africa and Oceania (Hausdorff et al., Clin Infect Dis 2000, 30:100-21). Furthermore, three highly penicillin-resistant strains were included, which represent the internationally spread clones Sp⁹-3, Sp¹⁴-3 and Sp²³-1 that account for a majority of penicillin-resistant pneumococci in day-care centers and hospitals (Sa-Leao et al., J Infect Dis 2000, 182:1153-60; Roberts et al., Microb Drug Resist 2001, 7:137-52). In 30 seconds, 100 U of Pal decreased the viable titer of the 15 strains of exponentially growing S. pneumoniae by Log₁₀ 4.0 cfu/ml (median, range 3.3-4.7) as compared to controls incubated with the enzyme buffer alone (FIG. 9). Pneumococci with intermediate (n=1) and high penicillin resistance (n=3) were killed at the same rate as penicillin sensitive strains (median (range) Log₁₀ 4.0 (3.7-4.7) vs. Log₁₀ 4.1 (3.3-4.7) cfu/ml, p=NS).

In addition, the capsule-deficient laboratory strain R36A and the mutant Lyt 4-4, deficient in capsule and lacking the major pneumococcal autolysin LytA, showed identical susceptibility to Pal as the clinical pneumococcal isolates (decrease of Log₁₀ 4.2 and 3.9 cfu/ml, respectively, p=NS). The latter results suggest that the pneumococcal capsule does not interfere with the enzyme's access to the cell wall and that autolysin does not contribute significantly to cell lysis caused by Pal.

One hundred units of Pal also killed exponentially growing S. oralis and S. mitis, but at a significantly lower rate (Log₁₀ 0.8 and Log₁₀ 0.23 cfu/ml, respectively, p<0.05). Both strains are known to incorporate choline in their cell walls and therefore provide a binding site for the enzyme (Gillespie et al., Infect Immun. 1993, 61:3076-7). The remaining oral streptococcal strains were unaffected with enzyme concentrations as high as 10,000 U/ml and up to 10 min of exposure.

Table 2 discloses bacterial strains tested for susceptibility to Pal. R indicates resistant; I indicates intermediate; and S indicates susceptible. Source 1 indicates Alexander Tomasz of The Rockefeller University, New York, N.Y.; source 2 indicates Paul Kohlenbrander of National Institute of Dental and Craniofacial Research, Bethesda, Md.; and source 3 indicates Ivo Van de Rijn of Wake Forest University, Winston-Salem, N.C.

TABLE 2
Bacterial strains tested for susceptibility to Pal

| | | Capsular | Susceptibility to | Clonal | |
|---|---|---|---|---|---|
| Species | Strain | group/type | Penicillin | type | Source |
| S. pneumoniae | DCC 1355 | 19F | S | | 1 (19) |
| S. pneumoniae | DCC 1335 | 9V | R | Sp⁹-3 | 1 |
| S. pneumoniae | DCC 1420 | 23F | R | Sp²³-1 | 1 |
| S. pneumoniae | DCC 1476 | 15 | I | | 1 |
| S. pneumoniae | DCC 1490 | 14 | S | | 1 |
| S. pneumoniae | DCC 1494 | 14 | R | Sp¹⁴-1 | 1 |
| S. pneumoniae | DCC 1714 | 3 | S | | 1 |
| S. pneumoniae | DCC 1808 | 24 | S | | 1 |
| S. pneumoniae | DCC 1811 | 11 | S | | 1 |
| S. pneumoniae | DCC 1850 | 6B | S | | 1 |
| S. pneumoniae | AR 314 | 5 | S | | 1 |
| S. pneumoniae | AR 620 | 1 | S | | 1 |
| S. pneumoniae | GB 2017 | 18 | S | | 1 |
| S. pneumoniae | GB 2092 | 4 | S | | 1 |
| S. pneumoniae | GB 2163 | 10 | S | | 1 |
| S. pneumoniae | R36A | | | | 1 (31) |
| S. pneumoniae | Lyt4-4 | | | | 1 (32) |
| S. gordonii | PK 2565 | | | | 2 |
| S. mitis | J 22 | | | | 2 |
| S. mutans | OMZ 175 | | | | 3 |
| S. oralis | H 1 | | | | 2 |
| S. salivarius | ATCC 27945 | | | | 2 |

R, resistant; I, intermediate; S, susceptible.
1, Alexander Tomasz, The Rockefeller University, New York, NY.
2, Paul Kohlenbrander, National Institute of Dental and Craniofacial Research, Bethesda, MD.
3, Ivo Van de Rijn, Wake Forest University, Winston-Salem, NC.

S. pneumoniae, including the R36A and Lyt 4-4 mutants, in stationary phase were more resistant to the lethal action of Pal. Nevertheless, exposure to 10,000 U/ml resulted in killing of Log₁₀ 3.0 cfu/ml (median, range 3.0-4.0) in 30 sec. The mechanism responsible for the decrease in susceptibility to hydrolysis by Pal in non-growing pneumococci is likely to be a change in the cell wall structure (Tuomanen and Tomasz, Scand J Infect Dis 1991, Suppl.74:102-12), such as an increase in peptidoglycan cross-linking.

Electron microscopy of S. pneumoniae serogroup 14 exposed to only 50 U/ml of Pal for 1 minute, revealed protrusions of the cell membrane and the cytoplasm through single breaks in the cell wall, which appeared predominantly near the septum of the dividing diplococci. After 5 min, empty cell walls remained, retaining their original shape, indicating that digestion of amide bonds in a restricted location within the cell wall is sufficient for cell death.

To determine if repeated exposure to low concentrations of Pal enzyme is able to select for resistant S. pneumoniae, strain DCC 1490 was grown on blood agar plates and exposed to low concentrations of enzyme (<1 U). Colonies at the periphery of a clearing zone were picked, grown to logarithmic phase, streaked on a fresh plate and re-exposed to Pal. Sixteen rounds of exposure did not result in decrease of susceptibility to Pal when compared to the unexposed strain using the in vitro killing assay (p=NS), demonstrating that resistance to Pal occurs at a very low frequency. It has been shown that the cell wall receptor for Pal as well as other pneumococcal phage lytic enzymes is choline, a molecule that is necessary for pneumococcal viability (Sheehan et al., Mol Microbiol 1997, 25:717-25; Lopez et al., Microb Drug Resist 1997, 3:199-211; Tomasz, Science 1967, 157:694-7). During a phage's association with bacteria over the millennia, to avoid being trapped inside the host, the binding domain of lytic enzymes has evolved to target a unique and essential molecule in the bacterial cell wall, making resistance to these enzymes a rare event.

### Example 3

### Identification of the Binding Receptor for the PlyG Enzyme on Bacillus RSVF

PlyG lysin, which was isolated from the γ phage of Bacillus anthracis, specifically kills B. anthracis and other members of the B. anthracis ‘cluster’ of bacilli both in vitro and in vivo (Schuch et al., Nature 2002, 418:884-889). The bacterial molecule(s) and pathway in B. anthracis conferring this specific susceptibility to the PlyG lysis has not been previously identified. Identification of such molecules and pathways provides the possibility for new, specific antibiotics for the treatment and prevention of anthrax to be developed.

The polysaccharide component of B. anthracis cell wall has been shown to contain galactose, N-acetylglucosamine and N-acetylmannosamine in an approximate moral ratio of 3:2:1 (Ekwunife et al., FEMS Microbiol Lett. 1991, 15:257-62; Fouet and Mesnage, Curr Top Microbiol Immunol. 2002, 271:87-113). To identify whether any of these polysaccharides might be involved in PlyG binding to the cell wall of B. anthracis, competition assays testing for the ability of each of these polysaccharides to block the ability of PlyG to kill the bacilli were performed. The present example demonstrates that N-acetylglucosamine is a receptor for the PlyG lysin. Thus, N-acetylglucosamine and the N-acetylglucosamine synthetic pathway should be targeted for antibacterial development.

### Methods

It is known that γ phages infect most B. anthracis isolates, including some rare Bacillus cereus strains that could represent B. anthracis cured of its virulence plasmid. Isolates of RSVF1, which is a streptomycin-resistant B. cereus strain #4342 from the American Type Culture Collection, and B. anthracis are monomorphic at multiple allozyme loci, and therefore are part of the same highly related cluster of isolates within the B. cereus lineage (Schuch et al., Nature 2002, 418:884-889). We have previously shown RSVF1 was sensitive to γ phage and display several other features typical of B. anthracis (Schuch et al., Nature 2002, 418:884-889). In addition, we have previously shown that RSVF1 was the only B. cereus strain that was as sensitive to PlyG killing as a diverse set of B. anthracis isolates (Schuch et al., Nature 2002, 418:884-889). Therefore, RSVF1 is used in the present example as the representative of the γ-phage-sensitive B. anthracis cluster of B. cereus.

The indicated concentration of N-acetylglucosamine was added to 1 ml exponential phase RSVF1 in 50 mM Tris buffer. PlyG lysin (5 g) was then added in 1 ml of 50 mM Tris buffer and incubated for 15 min at 37° C. The cells were then washed 2 times with buffer and plated. Bacterial counts (CFU ml⁻¹) were recorded.

### Results

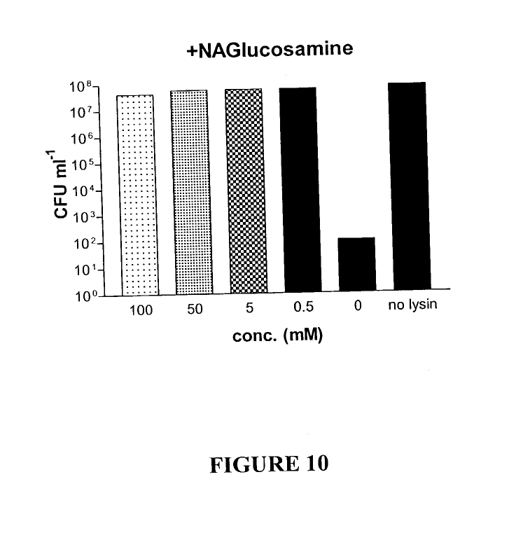

To identify a binding receptor for the PlyG lysin in the cell wall of bacillus RSVF, we used a competition assay in which the ability of sugars found in the cell wall of these bacteria (galactose, N-acetylglucosamine and N-acetylmannosamine) to block or decrease the killing ability of PlyG was tested. Neither galactose nor N-acetylmannosamine blocked PlyG's killing ability for the bacilli. However, as demonstrated in FIG. 10, as little as 0.5 mM of N-acetylglucosamine could block the lethal action of the PlyG, suggesting that this sugar plays a major role in the binding of PlyG in the cell wall. Thus, a wall complex containing N-acetylglucosamine would be a receptor substrate.

We have previously shown that repeated expose to low or high PlyG concentrations did not result in spontaneously generated mutants resistant to PlyG (Schuch et al., Nature 2002, 418:884-889). In addition, although methane-sulphonic acid ethyl ester (EMS) mutagenesis of RSVF1 resulted in 1,000-fold and 10,000-fold increases in novobiocin and streptomycin resistance, EMS mutagenesis did not result in PlyG-resistant mutants. (See Table 2 on page 887 of Schuch et al., Nature 2002, 418:884-889). The lack of PlyG-resistant mutants suggests that resistance to antimicrobials designed to target N-acetylglucosamine (or a complex containing N-acetylglucosamine) or molecules in its biosynthetic pathway will be rare. Therefore, inhibitors blocking N-acetylglucosamine and components of the N-acetylglucosamine biosynthetic pathway will be especially important new anti-infectives in the control of anthrax. The N-acetylglucosamine biosynthetic pathway is well described (Park, J Bacteriol, 2001: 183: 3842-7; Plumbridge J, and Vimr E., J. Bacteriol., 1999: 181:47-54; Skarzynski et al., Structure, 1996: 4:1465-74) and one skilled in the art could readily identify other targets in this biosynthetic pathway.

### Discussion

Bacteriophage lysin binding receptors, polyrhamnose on Streptococci for C₁ bacteriophage lysin and N-acetylglucosamine on B. anthracis for PlyG, are identified herewith. These bacterial molecules are essential for viability of the bacterial cell and are, thus, excellent targets for the development of antimicrobials. It is important to note that other bacterial cell wall molecules may also be involved in lysin binding and therefore may also serve as the basis for antimicrobial development. For example, PlyG may recognize other molecules in addition to, or complexed with, N-acetylglucosamine and these molecules may also serve as the starting point for the development of anti-infectives.

Antibiotics developed against bacterial pathways identified using bacteriophage lysins like C₁ bacteriophage lysin Pal, and PlyG have tremendous potential for the antimicrobial armamentarium. The results presented herewith support that by exploiting the fact that bacteriophage have evolved to target essential pathways of pathogenic bacteria, pathways essential for pathogenic bacteria can be identified and thus, unique essential pathway-targeting antibiotics can be developed. The essential nature of lysin receptor molecules in the bacterial cell makes them particularly attractive targets for the development of antimicrobials because resistance to these antimicrobials should be very rare.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A method for identifying a bacterial molecule that is essential for bacterial viability, which method comprises identifying a bacterial molecule that binds a bacteriophage lysin binding domain.

2. The method of claim 1, wherein the bacteriophage lysin binding domain comprises a lytically active lysin.

3. The method of claim 2, wherein binding of the bacteriophage lysin to the bacterial molecule results in bacterial lysis or cell wall component lysis.

4. The method of claim 1, wherein the bacteriophage lysin binding domain is from a C₁ bacteriophage lysin.

5. The method of claim 4, wherein the bacteria is an A, C, or E streptococcus.

6. The method of claim 5, wherein the bacterial molecule comprises a polyrhamnose.

7. The method of claim 1, wherein the bacteriophage lysin binding domain is from PlyG.

8. The method of claim 4, wherein the bacteria is a Bacillus anthracis.

9. The method of claim 5, wherein the bacterial molecule comprises an N-acetylglucosamine.

10. The method of claim 1, wherein the method comprises pretreating cells with an enzyme to identify targets which display disruptions in binding interaction.

11. The method of claim 10, wherein the enzyme is selected from proteases or glycosidases, or other cell wall digestive enzymes.

12. The method of claim 10, wherein the enzyme is selected from the group consisting of Pronase, protease K, trypsin, L-rhamnosidase, glycosidase or Ac-hexosaminidase.

13. The method of claim 1, which comprises detecting inhibition of binding of the bacteriophage lysin binding domain to the bacterial molecule in the presence of a competitive inhibitor in solution or suspension.

14. The method of claim 13, wherein the competition experiment comprises contacting the bacterial molecule with lysin in buffer containing a monosaccharide, cell wall carbohydrate extract, or lectin.

15. The method of claim 14, wherein the cell wall carbohydrate extract is an extracted group A Streptococcus carbohydrate.

16. The method of claim 14, wherein the cell wall carbohydrate extract is an extracted Bacillus anthracis carbohydrate.

17. A method for identifying a gene for a product in an essential pathway for bacterial viability, which method comprises determining whether mutating a gene results in a defect in a bacterial molecule that binds a bacteriophage lysin binding domain, wherein mutation of such a gene indicates that the gene for a product is in an essential pathway.

18. The method of claim 17, wherein the gene is involved in synthesis of the bacterial molecule.

19. The method of claim 17, wherein the defect in the bacterial molecule is loss of bacteriophage lysin binding activity.

20. The method of claim 17, wherein the bacteriophage lysin is bacteriophage C₁ lysin.

21. The method of claim 20, wherein the bacterial molecule comprises a polyrhamnose.

22. The method of claim 21, wherein the polyrhamnose is an A, C, or E streptococcus polyrhamnose.

23. The method of claim 17, wherein the bacteriophage lysin is PlyG.

24. The method of claim 20, wherein the bacterial molecule comprises an N-acetylglucosamine.

25. The method of claim 21, wherein the N-acetylglucosamine is a Bacillus anthracis N-acetylglucosamine.

26. A method for identifying a lead molecule effective as an antibiotic, which method comprises contacting a gene product of an essential pathway for bacterial viability, which pathway involves the biosynthesis of a bacterial molecule that contains a bacteriophage lysin binding domain, with a candidate molecule and determining whether the candidate molecule inhibits the essential pathway, wherein a candidate molecule that inhibits the essential pathway is a lead molecule effective as an antibiotic.

27. The method of claim 26, wherein the gene product is involved in synthesis of the bacterial molecule.

28. The method of claim 26, wherein the candidate compound that inhibits the essential pathway causes loss of bacteriophage lysin binding activity.

29. The method of claim 26, wherein the bacteriophage lysin is bacteriophage C₁ lysin.

30. The method of claim 29, wherein the bacterial molecule comprises a polyrhamnose.

31. The method of claim 30, wherein the polyrhamnose is an A, C, or E streptococcus polyrhamnose.

32. The method of claim 26, wherein the bacteriophage lysin is PlyG.

33. The method of claim 29, wherein the bacterial molecules comprises an N-acetylglucosamine.

34. The method of claim 30, wherein the N-acetylglucosamine is a Bacillus anthracis N-acetylglucosamine.
